(19) United States
(12) Patent Application Publication
(10) Pub. No.: US 2024/0266050 A1
(43) Pub. Date: Aug. 8, 2024

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Taisuke IWAMURA, Utsunomiya (JP); Hiroshizu MORISHIMA, Utsunomiya (JP); Kazutoshi YANAGIDA, Otawara (JP); Takayuki TANEMOTO, Otawara (JP)

(73) Assignee: CANON MEDICAL SYSTEMS CORPORATION, Otawara-shi (JP)

(21) Appl. No.: 18/426,433

(22) Filed: Jan. 30, 2024

(30) Foreign Application Priority Data
Feb. 2, 2023 (JP) 2023-014672

(51) Int. Cl. G16H 50/20; G16H 15/00

(52) U.S. Cl. CPC G16H 50/20; G16H 15/00

(57) ABSTRACT

A medical information processing device of an embodiment includes processing circuitry. The processing circuitry acquires a plurality of verification data sets including medical data, result data obtained by inputting the medical data to a trained model, and true/false data regarding the result data. The processing circuit identifies a target verification data set suitable for evaluating a performance required for a trained model that outputs the result data in response to input of the medical data among the plurality of verification data sets on the basis of the relationship between a first trained model that outputs the result data in response to input of the medical data and the verification data sets.

### CROSS-REFERENCE TO RELATED APPLICATION

The present application claims priority based on Japanese Patent Application No. 2023-014672, filed Feb. 2, 2023, the content of which is incorporated herein by reference.

### FIELD

Embodiments disclosed in this specification and drawings relate to a medical information processing device, a medical information processing method, and a storage medium.

### BACKGROUND

In recent years, machine learning technology using artificial intelligence (AI) has been used in a variety of systems, and machine learning technology has also been widely applied to the medical industry. Trained models such as analysis applications used for machine learning are updated through additional learning to improve the accuracy on a regular basis.

In a case where a trained model is updated to improve the accuracy, the direction of the update may not match the user's needs or operations, and in some cases, updating the trained model may result in deterioration. In order to avoid cases in which updating a trained model results in deterioration, it is necessary to update the trained model to suit the user's needs and operations.

### BRIEF DESCRIPTION OF THE DRAWINGS

FIG. 1 is a block diagram showing an example of a configuration of an in-hospital system of an embodiment.

FIG. 2 is a block diagram showing an example of a configuration of a medical information processing device of an embodiment.

FIG. 3 is a diagram showing an example of the content of a trained model.

FIG. 4 is a diagram showing an example of the content of a verification data set.

FIG. 5 is a diagram showing an example of the content of determination criterion data.

FIG. 6 is a diagram showing the number of verification data sets included in the verification data set.

FIG. 7 is a flowchart showing an example of processing in the medical information processing device of the embodiment.

FIG. 8 is a flowchart showing an example of processing in the medical information processing device of the embodiment.

### DETAILED DESCRIPTION

Hereinafter, a medical information processing device, a medical information processing method, and a storage medium according to embodiments will be described with reference to the drawings.

A medical information processing device of an embodiment is used, for example, as a part of an in-hospital system provided within a hospital. The medical information processing device of the embodiment includes processing circuitry. The processing circuitry acquires a plurality of verification data sets including medical data, result data obtained by inputting the medical data to a trained model, and true/false data regarding the result data. The processing circuit identifies a target verification data set suitable for evaluating a performance required for a trained model that outputs the result data in response to input of the medical data among the plurality of verification data sets on the basis of the relationship between a first trained model that outputs the result data in response to input of the medical data and the verification data sets. Accordingly, it is possible to appropriately update a trained model.

FIG. 1 is a block diagram showing an example of a configuration of an in-hospital system 1 according to an embodiment. The in-hospital system 1 of the embodiment includes, for example, a hospital information system (hereinafter, HIS) 10, a radiology information system (hereinafter, RIS) 20, and a medical image diagnostic device (modality) 30, a picture archiving and communication system (PACS) 40, and a medical information processing device 100.

The HIS 10 is a computer system that provides operational support in a hospital. Specifically, the HIS 10 includes various subsystems. Various subsystems include, for example, an electronic medical record system, a medical accounting system, a medical reservation system, a hospital visit reception system, and an admission/discharge management system.

The HIS 10 is, for example, a computer such as a server device or a client terminal which includes a processor such as a central processing unit (CPU), a memory such as a read only memory (ROM) or a random access memory (RAM), a display, an input interface, and a communication interface.

A user inputs or refers to information regarding a patient using an electronic medical record system included in the HIS 10. The user issues an image examination order to the HIS 10. The HIS 10 transfers order information corresponding to the image examination order to other systems such as the RIS 20.

The RIS 20 is a computer system that provides operational support in an image diagnosis department. The RIS 20 performs association of reservation information with examination equipment, management of examination information, and the like in addition to reservation management of image examination orders in cooperation with the HIS 10. The RIS 20 includes, for example, a computer such as a server device or a client terminal which includes a processor such as a CPU, a memory such as a ROM or a RAM, a display, an input interface, and a communication interface.

The modality 30 performs image-capturing (imaging) according to imaging conditions (imaging protocol) determined on the basis of an image examination instruction or the like, for example. Examples of the modality 30 include an X-ray computed tomography device, an X-ray diagnostic device, a magnetic resonance imaging device, an ultrasound diagnostic device, a nuclear medical diagnostic device, and the like. The modality 30 is operated by, for example, an operator such as a doctor (radiologist) or a medical radiology technician. Medical images (image data) generated by image-capturing performed by the modality 30 are transmitted to the PACS 40.

The PACS 40 is a computer system that receives medical images transmitted by the modality 30 and stores the medical images in a database. The PACS 40 transmits (transfers) medical images stored in the database in response to a request from a client. The PACS 40 includes a server computer including a processor such as a CPU, a memory such as a ROM or a RAM, a display, an input interface, and a communication interface. Information regarding patients to be imaged and imaging are associated with medical images stored in the PACS 40 as supplementary information. The supplementary information includes information such as patient IDs, examination IDs, and imaging conditions (imaging protocol) in a format in accordance with the digital imaging and communication in medicine (DICOM) standards, for example.

The configuration of the in-hospital system 1 is not limited to the above. The in-hospital system 1 may include, for example, an image interpretation report creation device or the like. Moreover, some elements of the in-hospital system 1 may be integrated. For example, the HIS 10 and the RIS 20 may be integrated into one system.

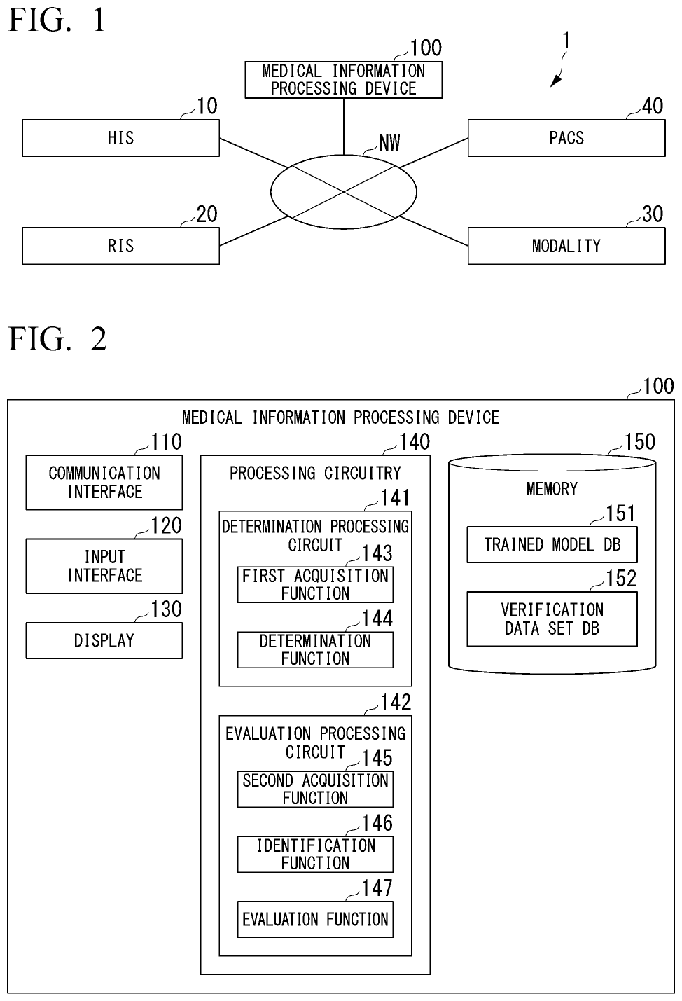

FIG. 2 is a block diagram showing an example of a configuration of the medical information processing device 100 according to the embodiment. The medical information processing device 100 includes, for example, a communication interface 110, an input interface 120, a display 130, processing circuitry 140, and a memory 150. The communication interface 110, the input interface 120, and the display 130 in the medical information processing device 100 are provided separately from the communication interface, the input interface, and the display included in the HIS 10, but these components may be common.

The communication interface 110 communicates with external devices such as the RIS 20, the modality 30, and the PACS 40 via a network NW such as a local area network (LAN). The communication interface 110 includes, for example, a communication interface such as a network interface card (NIC). The network NW may include the Internet, a cellular network, a Wi-Fi network, a wide area network (WAN), and the like instead of or in addition to a LAN.

The input interface 120 includes, for example, a mouse, a keyboard, a trackball, a switch, a button, a joystick, a touch panel, and the like. The input interface 120 may be, for example, a user interface that receives audio input, such as a microphone. In a case where the input interface 120 is a touch panel, the input interface 120 may also have the display function of the display 130.

Note that in this specification, the input interface is not limited to one that includes physical operation parts such as a mouse and a keyboard. For example, examples of the input interface include electrical signal processing circuitry that receives an electrical signal corresponding to an input operation from an external input apparatus provided separately from the device and outputs the electrical signal to a control circuit.

The input interface 120 receives various input operations from a doctor or the like, converts the received input operations into electrical signals, and outputs the electrical signals to the processing circuitry 140. For example, a doctor or the like performs a selection input operation to select a trained model (pre-trained machine learning model) for discovering a specific disease (e.g. cerebral infarction, hereinafter, a specific disease) in a patient (hereinafter, a target patient) for which medical image data has been captured. For example, in a case where a selection input operation is performed by a doctor or the like, the input interface 120 generates selection information according to the input operation. The input interface 120 outputs the generated selection information to the processing circuitry 140. Medical image data is an example of medical data.

For example, in evaluating the performance of a trained model, a doctor or the like performs an operation of inputting a required performance to identify a trained model to be evaluated and a performance required for the trained model to be evaluated. For example, in a case where the operation of inputting a required performance is performed by a doctor or the like, the input interface 120 generates required performance information according to the input operation. The input interface 120 outputs the generated required performance information to the processing circuitry 140. Evaluation of the performance of a trained model will be further described later.

The display 130 displays various types of information. For example, the display 130 displays images generated by the processing circuitry 140, a graphical user interface (GUI) for receiving various input operations from an operator, and the like. For example, the display 130 is a liquid crystal display (LCD), a cathode ray tube (CRT) display, an organic electroluminescence (EL) display, or the like.

The processing circuitry 140 includes, for example, a determination processing circuit 141 and an evaluation processing circuit 142. The processing circuitry 140 realizes these functions by, for example, a hardware processor (computer) executing a program stored in the memory (storage circuit) 150.

A hardware processor is, for example, circuitry such as a CPU, a graphics processing unit (GPU), an application specific integrated circuit (ASIC), a programmable logic device (for example, a simple programmable logic device (SPLD) or a complex programmable logic device (CPLD)) or a field programmable gate array (FPGA).

Instead of storing the program in the memory 150, the program may be directly incorporated into the circuit of the hardware processor. In this case, the hardware processor realizes the functions by reading and executing the program incorporated into the circuit. The aforementioned program may be stored in advance in the memory 150 or may be stored in a non-transitory storage medium such as a DVD or a CD-ROM and installed in the memory 150 from the non-transitory storage medium when the non-transitory storage medium is set in a drive device (not shown) of the medical information processing device 100.

The hardware processor is not limited to being configured as a single circuit, and may be configured as a single hardware processor by combining a plurality of independent circuits to realize each function. Further, a plurality of components may be integrated into one hardware processor to realize each function. Although the hardware processor, the memory, and the like in the medical information processing device 100 are provided separately from the hardware processor, the memory, and the like in the HIS 10, these components may be common.

The memory 150 stores a trained model database (hereinafter, a DB) 151 and a verification data set DB 152. The trained model DB 151 is a DB that includes a plurality of trained models. The verification data set DB 152 is a DB that includes a plurality of verification data sets. A verification data set is a set of medical image data, result data, and ground truth data.

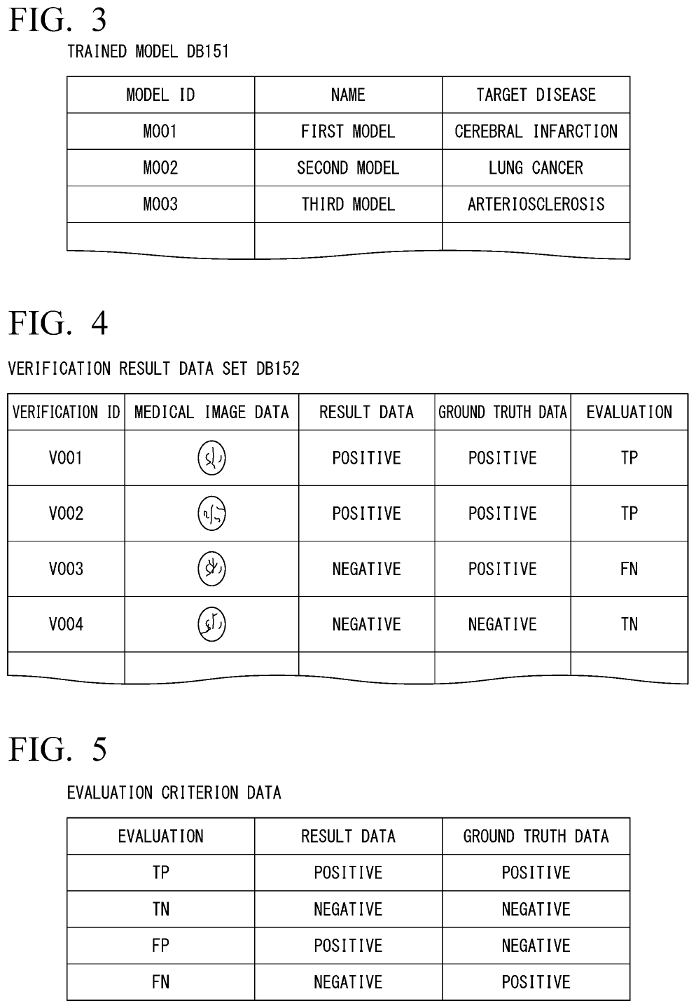

FIG. 3 is a diagram showing an example of the content of the trained model DB 151. The trained model DB 151 includes data of a plurality of trained models. A model ID and a name are assigned to each trained model. For example, a model ID of “M001” is assigned to a first model.

The trained models included in the trained model DB 151 are models for diagnosing a target patient. A trained model determines whether or not a specific disease is detected in the target patient (positive or negative) as diagnosis of the target patient, for example. Whether or not a specific disease is discovered in the target patient is determined, for example, based on whether or not medical image data obtained by imaging the target patient is image data including a specific disease.

A corresponding specific disease is set in each trained model. A trained model may not have a specific disease set therein and may be, for example, a model that identifies the content (type) of a disease. When medical image data is input as input data, the trained model outputs a result of determining whether medical image data corresponds to a specific disease as output data.

The trained model is updated using the result of determining whether or not the medical image data corresponds to a specific disease using the trained model, and ground truth data obtained from, for example, a finding report or the like. The trained model may be updated, for example, every time a determination result is output or every time a predetermined number of determination results are output.

FIG. 4 is a diagram showing an example of the content of the verification data set DB 152. The verification data set DB 152 includes a plurality of verification data sets. A verification ID and a determination result are assigned to each verification data set. The verification data set DB 152 is created for each of a plurality of trained models.

For example, medical image data is data of a medical image captured by the modality 30 included in the in-hospital system 1. The medical image data may be data provided by the PACS 40 or may be data provided by an external device installed outside the in-hospital system 1.

For example, result data is data regarding a diagnosis result and is data indicating a result obtained by inputting medical image data to a trained model. Ground truth data is data indicating presence or absence (positive or negative) of a specific disease set in a trained model. The ground truth data is, for example, data based on a doctor's finding. The ground truth data is included in a finding report created by a doctor, for example. A determination result is determined on the basis of determination criterion data. Ground truth data is an example of true/false data.

FIG. 5 is a diagram showing an example of the content of determination criterion data. The determination criterion data is true positive “TP” when result data is “positive” and ground truth data is “positive” and true negative “TN” when the result data is “negative” and the ground truth data is “negative.” The determination criterion data is false positive “FP” when the result data is “positive” and the ground truth data is “negative” and false negative “FN” when the result data is “negative” and the ground truth data is “positive.”

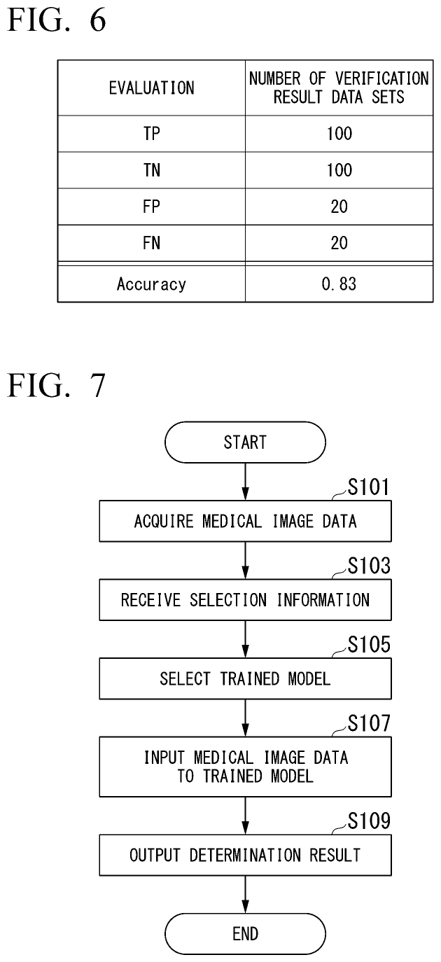

The verification data set DB 152 includes a plurality of pieces of verification data. FIG. 6 is a diagram showing the number of verification data sets included in the verification data set DB 152. The verification data set DB 152 shown in FIG. 6 includes 100 data sets with a determination result of “TP,” 100 data sets with a determination result of “TN,” 20 data sets with a determination result of “FP,” and 20 data sets with a determination result of “FN.” As a result, an accuracy rate of a trained model for which the verification data set DB 152 has been created is approximately 0.83 (83%).

The determination processing circuit 141 in the processing circuitry 140 is a circuit for determining presence or absence of a specific disease using a trained model on the basis of medical image data captured by the modality 30 or the like. The determination processing circuit 141 includes, for example, a first acquisition function 143 and a determination function 144.

The first acquisition function 143 acquires medical image data captured by the modality 30. Here, the medical image data is image data for determining presence or absence of a specific disease. The medical image data may be medical image data other than medical image data captured by the modality 30.

The determination function 144 selects a trained model for determining the medical image data acquired by the first acquisition function 143 from the trained model DB 151 on the basis of input operation information transmitted by the input interface 120 in response to an input operation performed by a doctor or the like. The determination function 144 determines whether a specific disease is discovered in a target patient by inputting the medical image data acquired by the first acquisition function 143 to the selected trained model.

The evaluation processing circuit 142 in the processing circuitry 140 is a circuit for evaluating the performance of a trained model used by the determination processing circuit 141 to determine whether or not a specific disease is discovered in a target patient. The evaluation processing circuit 142 includes, for example, a second acquisition function 145, an identification function 146, and an evaluation function 147.

The second acquisition function 145 acquires a plurality of verification data sets, for example, in evaluating the performance of a trained model. The second acquisition function 145 further acquires a performance required for the trained model to be evaluated (hereinafter, a required model performance) and a trained model serving as a criterion for performance evaluation (hereinafter, a first trained model). The second acquisition function 145 is an example of an acquisition unit.

The identification function 146 identifies, for example, a verification data set (hereinafter, a target verification data set) suitable for evaluating the required model performance. The required model performance may be, for example, a performance that is highly likely to be determined as true positive or true negative (have a high correct detection rate), or a performance that is less likely to be determined as false positive or false negative (have a low false detection rate).

The required model performance obtained here is determined on the basis of required performance information transmitted by the input interface 120, for example. The target verification data set is, for example, a verification data set for which a result corresponding to the required model performance has been obtained. For example, in a case where the required model performance is highly likely to be true positive, a large number of verification data sets for which a determination result is true positive “TP” is identified as target verification data sets, or verification data sets for which all determination results are true positive “TP” are identified target verification data sets.

The evaluation function 147 designates a trained model (hereinafter, a second trained model) to be evaluated and evaluates the second trained model, for example. The second trained model is different from the first trained model. The evaluation function 147 evaluates the performance of the second trained model on the basis of the relationship between the first trained model and the target verification data set and the relationship between the second trained model and the target verification data set using uses the target verification data set identified by the identification function 146. The second trained model is, for example, a trained model that has undergone further learning from the first trained model. The evaluation function 147 is an example of an evaluation unit.

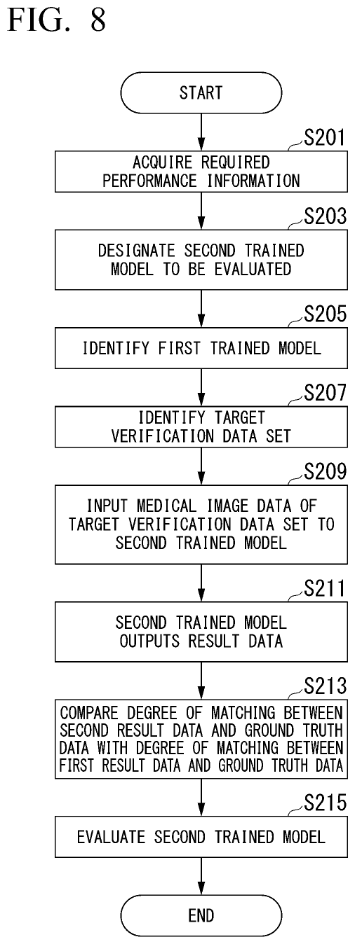

In the medical information processing device 100 of the first embodiment, processing of determining a specific disease in a target patient and processing of evaluating a trained model used for diagnosis are performed. These types of processing will be described below. Here, processing of determining a specific disease in a target patient will be described first. FIG. 7 and FIG. 8 are flowcharts showing examples of processing in the medical information processing device 100 of an embodiment.

FIG. 7 shows an example of processing of determining a specific disease in a target patient. The processing shown in the flowchart of FIG. 7 is mainly performed in the determination processing circuit 141, for example. First, the medical information processing device 100 acquires medical image data of a region to be diagnosed in the target patient, for example, the brain in a case where the specific disease is cerebral infarction through the first acquisition function 143 (step S101).

Subsequently, the first acquisition function 143 acquires selection information transmitted by a doctor or the like operating the input interface 120 (step S103). The selection information includes information on a trained model for use in diagnosis selected by a doctor or the like from the trained model DB 151.

Subsequently, the determination function 144 selects a trained model to be used for diagnosis on the basis of the selection information acquired by the first acquisition function 143 (step S105). Subsequently, the determination function 144 inputs the medical image data acquired by the first acquisition function 143 to the selected trained model (step S107).

In the determination function 144, the trained model outputs result data indicating a determination result when the medical image data has been used as input data (step S109). The determination function 144 causes the display 130 to display, for example, the determination result (“TP”, “FN”, or the like) indicated by the result data. In this manner, the medical information processing device 100 ends processing shown in FIG. 7.

Next, processing of evaluating a trained model used for diagnosis will be described. FIG. 8 shows an example of processing of evaluating a trained model used for diagnosis. The processing shown in the flowchart of FIG. 8 is mainly performed in the evaluation processing circuit 142, for example. First, the medical information processing device 100 acquires required performance information transmitted by a doctor or the like operating the input interface 120 through the second acquisition function 145 (step S201).

Subsequently, the identification function 146 designates a second trained model to be evaluated on the basis of the required performance information acquired by the second acquisition function 145 (step S203). Subsequently, the identification function 146 designates a first trained model on the basis of the designated second trained model (step S205). The identification function 146 designates, for example, a trained model immediately before the second trained model is updated as the first trained model. The identification function 146 may use another trained model, for example, a trained model before a plurality of second trained models are updated, or a known trained model as the first trained model.

Subsequently, the identification function 146 identifies target verification data sets on the basis of required performance information acquired by the second acquisition function 145 (step S207). The identification function 146 identifies the target verification data sets according to the performance required by the required performance information. For example, in the required performance information, in a case where the required model performance is increasing the possibility of true positives, a large number of verification data sets for which a determination result is true positive “TP” is identified as target verification data sets, or verification data sets for which all determination results are true positive “TP” are identified as target verification data sets.

In addition, in a case where the required model performance is increasing the possibility of true negatives, a large number of verification data sets for which a determination result is true negative “TN” is identified as target verification data sets, or verification data sets for which all determination results are true negative “TN” are identified as target verification data sets.

In addition, in a case where the required model performance is decreasing the possibility of false positive, verification data in which ground truth data of a verification data set for which a determination result is false positive “FP” is “negative” is generated, and a target verification data set including the generated verification data is identified as a target verification data set.

In addition, in a case where the required model performance is decreasing the possibility of false negative, verification data in which ground truth data of a verification data set for which a determination result is false negative “FN” is “positive” is generated, and a target verification data set including the generated verification data is identified as a target verification data set.

Subsequently, the evaluation function 147 inputs medical image data included in each of a plurality of target verification data sets to the second trained model in order to evaluate the second trained model using the target verification data sets identified by the identification function 146 (step S209). Subsequently, the second trained model to which the medical image data has been input outputs result data (hereinafter, second result data) (step S211).

Next, the evaluation function 147 collects result data (hereinafter, first result data) output by the first trained model from the verification data set DB 152 by inputting the medical image data obtained from the second result data to the first trained model. The evaluation function 147 refers to the verification data set DB 152 to obtain a degree of matching between the second result data and ground truth data and a degree of matching between the first result data and the ground truth data. The evaluation function 147 compares the degree of matching between the second result data and the ground truth data and the degree of matching between the first result data and the ground truth data (step S213). The second trained model is evaluated on the basis of a result of comparison between the degree of matching between the second result data and the ground truth data and the degree of matching between the first result data and the ground truth data (step S215).

For example, in a case where the degree of matching between the second result data and the ground truth data is higher than the degree of matching between the first result data and the ground truth data, the evaluation function 147 evaluates that the second trained model has higher performance than the first trained model. For example, in a case where the degree of matching between the second result data and the ground truth data is lower the degree of matching between the first result data and the ground truth data, the evaluation function 147 evaluates that the second trained model has lower performance than the first trained model. In this manner, the medical information processing device 100 ends the processing shown in FIG. 8.

The medical information processing device 100 of the embodiment identifies a target verification data set used at the time of evaluating a required model performance of a trained model generated by updating the first trained model on the basis of the relationship between the first trained model and the target verification data set in the evaluation function 147. Therefore, target verification data suitable for evaluating the required model performance of the second trained model can be identified, and thus the trained model can be updated appropriately.

Although the medical information processing device 100 includes the determination processing circuit 141 and the evaluation processing circuit 142 and performs determination processing and evaluation processing in the above-described embodiment, the medical information processing device 100 may include the evaluation processing circuit 142 without including the determination processing circuit 141. In this case, the medical information processing device 100 may be provided with a result of determining presence or absence of a specific disease and verification data sets by an external device.

Further, although the medical information processing device 100 is provided as a part of the in-hospital system 1 in the above-described embodiment, the medical information processing device 100 may be provided independently from the in-hospital system 1. In this case, the medical information processing device 100 may evaluate the second trained model using a verification data set generated within the hospital or may evaluate the second trained model using a verification data set provided from the outside of the hospital.

Further, selection of a trained model is performed on the basis of selection information according to a selection input operation performed by a doctor or the like in the above-described embodiment, but it may be performed by the medical information processing device 100 on the basis of various types of information such as the type of medical image data.

Further, although true/false data is ground truth data in the above-described embodiment, the true/false data may be misunderstanding data (incorrect answer data) or may include ground truth data and misunderstanding data. Further, although medical data is medical image data in the above-described embodiment, the medical data may be data other than medical image data. The medical data may be, for example, medical text data in which diagnosis results and the like are written.

According to at least one embodiment described above, it is possible to appropriately update a trained model by including an acquisition unit that acquires a plurality of verification data sets including medical data, result data obtained by inputting the medical data to a trained model, and true/false data regarding the result data, and an identification unit that identifies a target verification data set suitable for evaluating a performance required for a trained model that outputs the result data in response to input of the medical data among the plurality of verification data sets on the basis of the relationship between a first trained model that outputs the result data in response to input of the medical data and the verification data sets.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing device comprising processing circuitry,
wherein the processing circuitry is configured to:
acquire a plurality of verification data sets including medical data, result data obtained by inputting the medical data to a trained model, and true/false data regarding the result data; and
identify a target verification data set suitable for evaluating a performance required for a trained model that outputs the result data in response to input of the medical data among the plurality of verification data sets on the basis of a relationship between a first trained model that outputs the result data in response to input of the medical data and the verification data sets.

2. The medical information processing device according to claim 1, wherein the processing circuitry evaluates a performance of a second trained model on the basis of a relationship between the first trained model and the target verification data set and a relationship between the second trained model and the verification data set,
wherein the second trained model is a trained model different from the first trained model and is a machine learning model trained to output the result data in response to input of the medical data.

3. The medical information processing device according to claim 1, wherein the result data is data regarding a diagnosis result, and the performance required of the trained model includes a low false positive false detection rate of the diagnostic result.

4. The medical information processing device according to claim 3, wherein the processing circuitry identifies the target verification data set including the verification data set in which the result data is positive and the true/false data is negative.

5. The medical information processing device according to claim 1, wherein the result data is data regarding a diagnosis result, and the performance required of the trained model includes a low false negative false detection rate of the diagnostic result.

6. The medical information processing device according to claim 5, wherein the processing circuitry identifies the target verification data set including the verification data set in which the result data is negative and the true/false data is positive.

7. The medical information processing device according to claim 1, wherein the true/false data includes data based on a finding report.

8. The medical information processing device according to claim 1, wherein the medical data is medical image data.

9. A medical information processing method using a computer, the medical information processing method comprising:
acquiring a plurality of verification data sets including medical data, result data obtained by inputting the medical data to a trained model, and true/false data regarding the result data; and
identifying a target verification data set suitable for evaluating a performance required for a trained model that outputs the result data in response to input of the medical data among the plurality of verification data sets on the basis of a relationship between a first trained model that outputs the result data in response to input of the medical data and the verification data sets and a relationship between a second trained model that outputs the result data in response to input of the medical data and is different from the first trained model and the verification data sets.

10. A computer-readable non-transitory storage medium storing a program for causing a computer to execute:
acquiring a plurality of verification data sets including medical data, result data obtained by inputting the medical data to a trained model, and true/false data regarding the result data; and
identifying a target verification data set suitable for evaluating a performance required for a trained model that outputs the result data in response to input of the medical data among the plurality of verification data sets on the basis of a relationship between a first trained model that outputs the result data in response to input of the medical data and the verification data sets and a relationship between a second trained model that outputs the result data in response to input of the medical data and is different from the first trained model and the verification data sets.
